(19) United States
(12) Patent Application Publication
(10) Pub. No.: US 2024/0066160 A1
(43) Pub. Date: Feb. 29, 2024

(54) SHELF FOR UV-C DISINFECTION

(71) Applicant: SIGNIFY HOLDING B.V., EINDHOVEN (NL)

(72) Inventors: MARC ANDRE DE SAMBER, LOMMEL (BE); RENE MAARTEN ARJAN DRIESSENS, EINDHOVEN (NL)

(21) Appl. No.: 18/282,184

(22) PCT Filed: Mar. 9, 2022
(86) PCT No.: PCT/EP2022/056045

§ 371 Date: Sep. 14, 2023

(30) Foreign Application Priority Data
Mar. 18, 2021 (EP) 21163327.6

(51) Int. Cl. A61L 2/10; A61L 2/26

(52) U.S. Cl. CPC A61L 2/10; A61L 2/26

(57) ABSTRACT

The present invention relates to a shelf (10) for a UV-C disinfection chamber (12) comprising at least one UV-C light source (24) adapted to emit UV-C radiation (22) inside the UV-C disinfection chamber, the shelf comprising: a metal mesh shelf plate (14); and at least one optical element (26), wherein each optical element of the at least one optical element is adapted to locally modify the UV-C radiation relative to an object (28) supported by the shelf in conjunction with the optical element when the shelf is placed inside the UV-C disinfection chamber.

### FIELD OF THE INVENTION

The present invention relates to a shelf for a UV-C disinfection chamber. The present invention also relates to a UV-C disinfection chamber comprising at least one such shelf. The present invention also relates to a UV-C disinfection method

### BACKGROUND OF THE INVENTION

Shelves with a net structure are previously known in ultraviolet (UV) sterilization.

KR20160032068 (A) discloses an ultraviolet sterilizer comprising a main body exterior unit and a main body interior unit is equipped with a diffused reflection means which is an uneven panel formed as a polygon on the upper surface, the side surface, and the rear surface of the main body interior unit, and equipped with a diffused reflection means which is an uneven panel formed as a polypyramid on the lower surface, for evenly diffused-reflecting the ultraviolet rays emitted from the ultraviolet lamp to the inside of the main body interior unit. In addition, the sterilizer is sterilized by forming shelves with a net structure or a material of UV-penetrable glass, and evenly reaching the ultraviolet wavelength the inside and the outside of the target objects held on the shelves.

### SUMMARY OF THE INVENTION

The recent outbreak of the COVID-19 pandemic has urged the use of respirator masks as one of the options for personal and interpersonal protection. Related to the need for circularity and overall availability and logistics, it may be wished to re-use respirator masks several times. This is not obvious however, as one not only has to deal with decontamination/disinfection of the masks but also with preserving the filter properties and the shape of the masks after the decontamination. In case of re-use of masks by others (so not the original first user) and mass/industrial disinfection of masks, a thorough disinfection is required, eliminating not only viral material but also bacterial species (e.g. that might otherwise lead to bacterial pneumonia transmission or alike between persons).

The present inventors foresee that UV-C light (e.g. 254 nm, or below) applied in a human-safe way will be a worthwhile approach for respirator mask disinfection. Such procedure can (best) be done in a closed chamber that removes any light exposure and microbiological risk for the user.

However, the present inventors have also realized that when putting respirator masks inside a UV-C exposure chamber, it is likely that the irradiance levels that are experienced by the mask will not be homogenous. Specifically, the inside of cup-shaped masks may receive lower intensities of UV light. Achieving sufficient energy on that mask location would necessitate high overexposures, leading to potential damage to the mask material or additional handling by the masks such as flipping them inside-out. Also the workflow, #masks disinfected/time, would be low. To this end, it is an object of the present invention to overcome or at least alleviate one or more of these problems.

According to a first aspect of the invention, this and other objects are achieved by a shelf for a UV-C disinfection chamber comprising at least one UV-C light source adapted to emit UV-C radiation inside the UV-C disinfection chamber, the shelf comprising: a metal mesh shelf plate; and at least one optical element, wherein each optical element of the at least one optical element is adapted to locally modify the UV-C radiation relative to an object supported by the shelf in conjunction with the optical element when the shelf is placed inside the UV-C disinfection chamber.

The present invention is based on the understanding that by adding at least one such optical element to what otherwise could be a conventional metal mesh shelf used in a UV-C disinfection chamber, UV-C radiation may be concentrated on locations on the object that are otherwise under-exposed and hence would not be properly/sufficiently disinfected, for example the inside of cup-shaped respiratory masks. This without having to use high overexposures and/or additional handling of the masks such as flipping them inside-out. Hence objects like respiratory masks can efficiently be disinfected with homogenous irradiance levels and without damage caused by overexposure. The present shelf could for example be used in ONCE's BioShift® Pass-Through UV-C Chamber.

Apart from respirator masks, other assets/objects could be disinfected using the present invention. E.g. personal objects, such as smart phones or keys. Or reusable objects, such as contactless thermometers in medical, or handheld scanners and other shared tools in industrial or agriculture settings.

The at least one optical element may be at least one beam shaping element.

The at least one optical element may be arranged on the metal mesh shelf plate. Specifically, the at least one optical element may be placed on a (top) surface of the metal mesh shelf plate, which makes installation of the at least one optical element easy and flexible.

Alternatively, the at least one optical element may be fixedly incorporated in a metal mesh structure of the metal mesh shelf plate. This might be done in an open setting in the metal mesh structure, as done for open diamond setting in a ring. This may facilitate handling of the present shelf, such as placing the shelf in, and removing it from, the UV-C disinfection chamber.

The at least one optical element may be at least one refractive optical element. For example, the at least one optical element may comprise at least one substantially horizontally arranged diverging lens. That is, the diverging lens' optical axis is (substantially) perpendicular to the plane of the metal mesh shelf plate. This optical element may for example be used to diverge at least some of the UV-C radiation such that it accesses the concave inside of a respiratory mask positioned over the optical element. That is, a new UV-C light irradiance distribution may be provided to optimize exposure/increase the irradiance level of otherwise shadowed surfaces of the respiratory mask. The at least one diverging lens may for example be at least one biconcave lens.

In another example, the at least one optical element comprises at least one substantially vertically arranged diverging lens. That is, the diverging lens' optical axis is (substantially) parallel to the plane of the metal mesh shelf plate. This optical element may for example be used to diverge at least some of the UV-C radiation such that it may accesses for example a port or socket of a mobile phone or the like lying on the metal mesh shelf plate in the direction of that diverging lens' optical axis. In other words, this optical element may be used to reach ‘hidden’ sides inside e.g. the port of a phone. The at least one diverging lens may for example be at least one biconcave lens.

In a third example, the at least one optical element comprises at least one focusing element adapted to increase an UV-C irradiance level on the object supported by the shelf in conjunction with the focusing element. This optical element may e.g. be used for reaching a deep surface inside an object. The focusing element's optical axis may be (substantially) parallel to the plane of the metal mesh shelf plate, and the object may be positioned on the metal mesh shelf plate in the direction of the optical axis. The at least one focusing element may for example be at least one Fresnel lens or at least one plano-convex lens.

In other embodiments, the at least one optical element may comprise a box on the metal mesh shelf plate, wherein the box is adapted to contain said object, and wherein the box comprises at least one UV-C transparent side. The at least one UV-C transparent side may for example be made of quartz or fused silica. The box may serve as a mini environment for safe and secure handling of a contaminated sample. The box may be cube-shaped or rectangular cuboid-shaped (hence with six sides), for example. One (top) side of the box may serve as a lid. At least one side of the box may be missing, resulting in an open box.

At least one side of the box may have an optical function beyond UV-C transmission, such as re-direction of the UV-C radiation inside the box for better light distribution towards a (3D) object in the box that needs to be disinfected. This at least one side may for example be diverging or focusing, similar to the optical elements mentioned above. Using e.g. 3D printing (additive manufacturing) for making a mold that is next used for a melting process of at least this at least one side of the box, basically any shape and hence any type of irradiance optimization for a specific object that needs to be disinfected can be fabricated.

A side of the box may have enhanced UV-C transmission compared to other sides of the box. For example, a side of the box pointing away from the at least one UV-C light source and/or a reflective inner wall of the UV-C disinfection chamber may have the enhanced UV-C transmission, for (more) homogenous irradiation inside the box.

The metal mesh shelf plate may have a metal mesh structure of wires, wherein the metal mesh structure has at least one local area with less wires, and wherein the at least one optical element is arranged at the at least one local area with less wires. This may allow better access to UV-C radiation to the optical element(s) while maintaining sufficient mechanical strength of the metal mesh shelf plate. A local area may for example have no more than 0.5 times the wires compared to a corresponding ‘normal’ area of the metal mesh structure. Also the local area might be constructed such that the earlier described refractive optical element is supported at it edges only (for mechanical stability); ‘open setting’. Optionally the refractive optical element may be constructed such that it has additional mechanical elements as to better interface/overlap with the (very open) metal mesh structure of this embodiment.

The metal mesh shelf plate may have at least one visual (2D or 3D) and/or mechanical (3D) alignment feature next to the at least one optical element as at least one reference point for correctly positioning the object in conjunction with the optical element. This may ensure that the object is irradiated as best as possible. The mechanical alignment feature(s) could also be adapted to hold the object in the correct position.

According to a second aspect of the invention, there is provided a UV-C disinfection chamber, comprising: at least one UV-C light source adapted to emit UV-C radiation inside the UV-C disinfection chamber; and at least one shelf (e.g. a single shelf or a plurality of shelves, for example four shelves) according to the first aspect placed inside the UV-C disinfection chamber.

According to a third aspect of the invention, there is provided a UV-C disinfection method, comprising: providing a shelf according to the first aspect, wherein the shelf is placed inside a UV-C disinfection chamber; positioning an object to be disinfected on the shelf in conjunction with an optical element of the at least one optical element of said shelf; operating the UV-C disinfection chamber such that at least one UV-C light source emits UV-C radiation inside the UV-C disinfection chamber; and locally modifying, by the optical element, the UV-C radiation relative to said object. This aspect may exhibit the same or similar features and technical aspects as any one of the previous aspects, and vice versa.

Locally modifying the UV-C radiation may include diverging at least some of the UV-C radiation such that it homogenously accesses a concave or recessed part or portion of said object. The optical element may for example be a substantially horizontally arranged diverging lens, wherein the object is a respiratory mask positioned over the substantially horizontally arranged diverging lens with its concave inside facing the substantially horizontally arranged diverging lens.

Locally modifying the UV-C radiation may also include focusing at least some of the UV-C radiation onto said object such that an UV-C irradiance level on the object is increased (compared to a situation without the optical/focusing element).

It is noted that the invention relates to all possible combinations of features recited in the claims.

### BRIEF DESCRIPTION OF THE DRAWINGS

This and other aspects of the present invention will now be described in more detail, with reference to the appended drawings showing embodiment(s) of the invention.

FIG. 1 is a perspective view of a shelf for a UV-C disinfection chamber according to one or more embodiments of the present invention.

FIGS. 2a-d are partial side views of shelves with refractive optical elements according to embodiments of the present invention.

FIG. 3 is a side view of a shelf with boxes according to at least one other embodiment of the present invention.

FIG. 4 is a top view of a shelf having local areas with less wires.

FIG. 5 is a perspective view of a shelf having visual and/or mechanical alignment features.

FIG. 6 is a perspective view of a UV-C disinfection chamber according to an aspect of the present invention.

FIG. 7 is a flow chart of a method according to an aspect of the present invention.

As illustrated in the figures, the sizes of layers and regions may be exaggerated for illustrative purposes and, thus, may be provided to illustrate the general structures of embodiments of the present invention. Like reference numerals refer to like elements throughout.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and fully convey the scope of the invention to the skilled person.

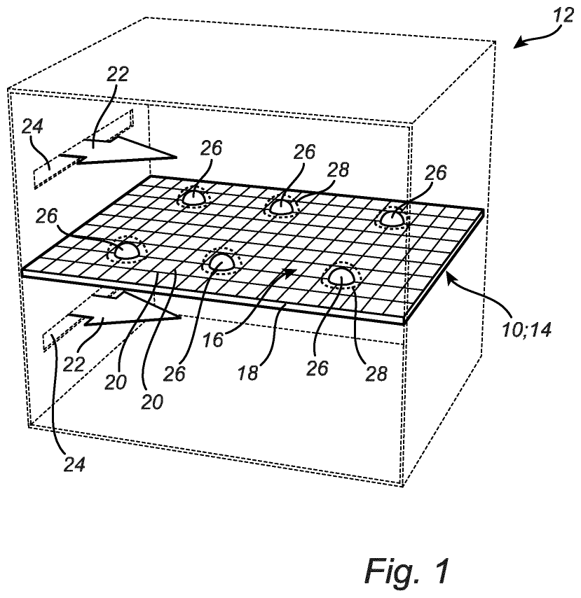

FIG. 1 is a perspective view of a shelf 10 for a UV-C disinfection chamber 12 according to one or more embodiments of the present invention.

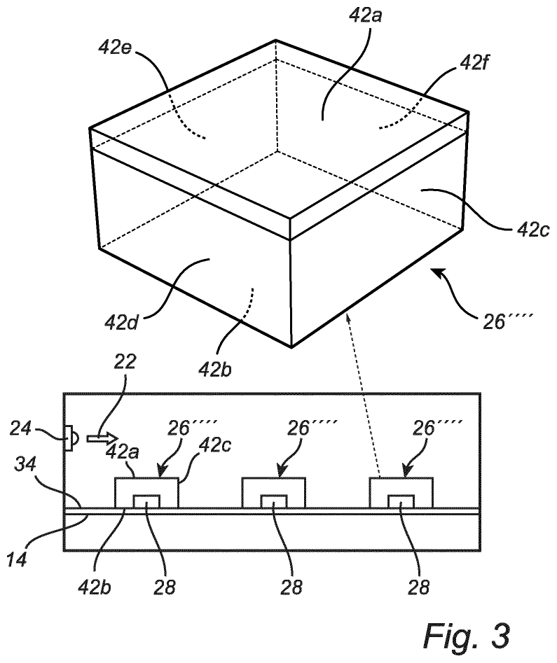

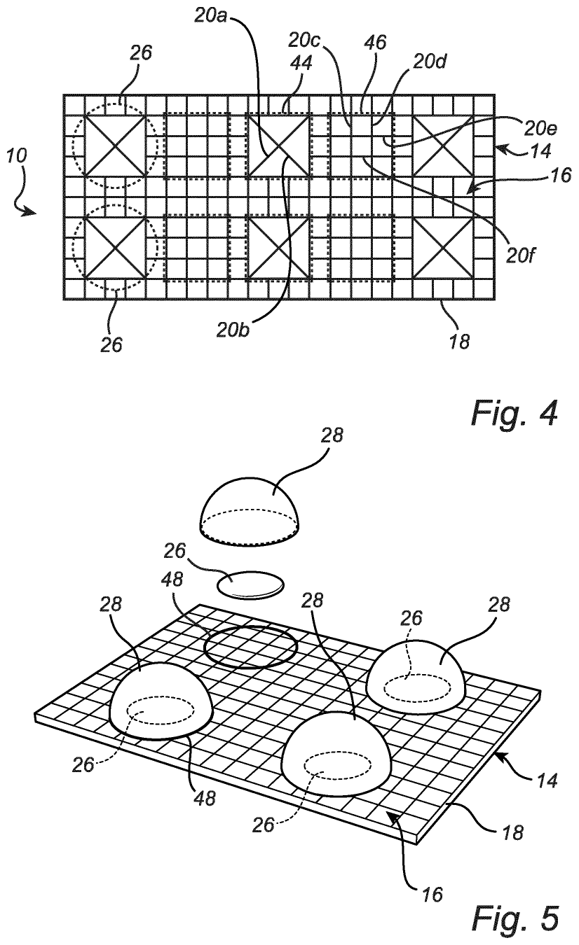

The shelf 10 comprises a metal mesh shelf plate 14. The metal mesh shelf plate 14 may be square or rectangular. The metal mesh shelf plate 14 could for example have a width and depth of at least 400 mm. The metal mesh shelf plate 14 may comprise a planar metal mesh structure 16 extending over most of the metal mesh shelf plate 14 apart from a peripheral (square or rectangular) frame 18. The metal mesh structure 16 may comprise corrugated crossing wires 20, see further FIGS. 4-5. The wires 20 may for example extend parallel relative to the frame 18, or diagonally relative to the frame 18. The metal mesh shelf plate 14 may for example be made of stainless steel. By means of the metal mesh structure 16, UV-C radiation 22 emitted by at least one UV-C light source 24 inside the chamber 12 may be (partly) transmitted through the metal mesh shelf plate 14.

The present UV-C radiation 22 may be in the range of 100 to 280 nm. Specifically, the present UV-C radiation 22 may be 254 nm, or below.

Different UV-C wavelengths of radiation may have different properties and thus may have different effects when used for disinfection (Table 1).

TABLE 1
Properties of different types of UV-C wavelength light

| | | | (Relative) | | | |
|---|---|---|---|---|---|---|
| | | | sterilization/ | | | |
| | | | inactivation | | | |
| | Short | Wavelength | effectiveness | | Safe | Ozone |
| Name | name | (nm) | Bacteria | Viruses | Radiation | generation |
| Near | Near | 230-280 | + | + | − | |
| ultraviolet | UV-C | | | | | |
| C | | | | | | |
| Far ultra- | Far UV | 190-230 | + | + | + | +/− |
| violet | | | | | | |
| Extreme | Extreme | 100-190 | + | + | − | + |
| ultraviolet | UV-C | | | | | |
| C | | | | | | |

Each UV-C type/wavelength range may have different benefits and/or drawbacks. Relevant aspects may include (relative) sterilization effectiveness and ozone production (as result of its radiation). Depending on an application a specific type of UV-C light or a specific combination of UV-C light types may be selected and provides superior performance over other types of UV light. Near UV-C may effectively kill bacteria and viruses. Far UV may also be effective in killing bacteria and viruses. Far-UV light may generate some ozone. Extreme UV-C may also be effective in killing bacteria and viruses. Extreme UV-C may generate ozone. Ozone may contribute to disinfection. Hence, in the table “+” for ozone production especially implies that ozone is produced which may be useful for disinfection applications, like in the present invention, but may be harmful for humans/animals if they are exposed to it.

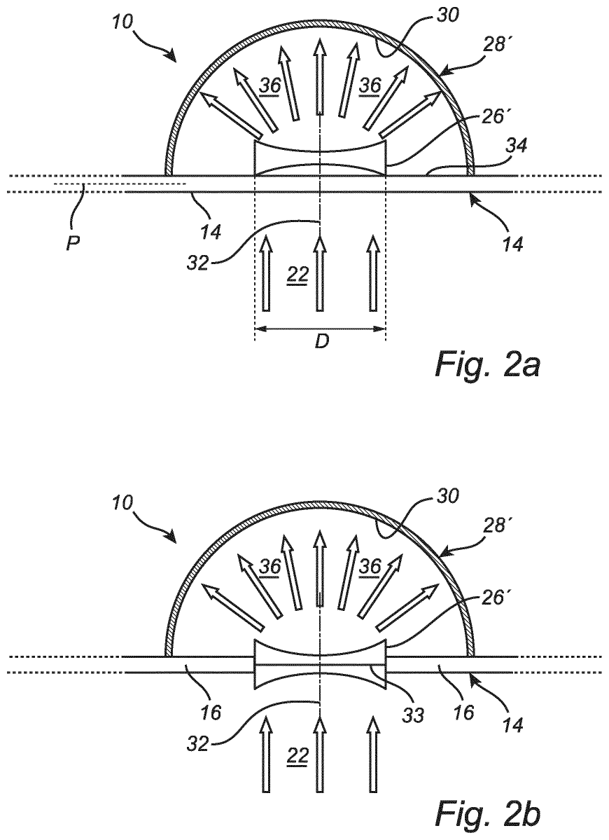

Returning to FIG. 1, the shelf 10 further comprises one or more (in FIG. 1 six) optical elements 26. Each optical element 26 is generally adapted to locally modify the UV-C radiation 22 relative to an object 28 supported by the shelf 10 in conjunction with the optical element 22 when the shelf 10 is placed inside the UV-C disinfection chamber 12. In this way, UV-C radiation may for example be re-directed to locations on the objects 28 that are otherwise under-exposed and hence would not be properly/sufficiently disinfected, for example the inside 30 of cup-shaped respiratory masks 28′ (see FIGS. 2a-b). The optical element(s) 26 can for example be made of quartz or fused silica. The optical elements 26 could be uniformly/regularly distributed across the metal mesh shelf plate 14, for example in a(n upright) square lattice point pattern as in FIG. 1 or in a diamond or diagonal square lattice point pattern.

In FIGS. 2a-b, the at least one optical element 26 is at least one refractive optical element, specifically at least one diverging lens 26′. The diverging lens 26′ is here a biconcave lens. The diameter D of the diverging lens 26′ may for be in the range of 3-10 cm, such as about 5 cm. The diverging lens 26′ in FIGS. 2a-b is horizontally arranged, such that its optical axis 32 is perpendicular to the plane P of the metal mesh shelf plate 14. In FIG. 2a, the diverging lens 26′ is placed on a top surface 34 of the metal mesh shelf plate 14. In another variant as shown in FIG. 2b, the diverging lens 26′ is incorporated in the metal mesh structure 16 of the metal mesh shelf plate 14. To this end, apart from the aforementioned parallel/diagonal wires 20, the metal mesh structure 16 could include rings 33 or hollow cylinders or clamps or the like holding the optical elements 26′ at fixed positions, without optical cross-talk. A respiratory mask 28′ may be positioned over the diverging lens 26′, with its concave inside 30 facing the diverging lens 26′. In use, the diverging lens 26′ may diverge at least some of the UV-C radiation 22 such that diverged UV-C radiation 36 accesses the inside 30 of the respiratory masks 28′, whereby the irradiance levels that are experienced by the respiratory masks 28′ may be more homogenous than without the diverging lens 26′.

Another embodiment illustrated in FIG. 2c is similar to that of FIG. 2a, but here a diverging lens 26″ is vertically arranged on the top surface 34, such that its optical axis 32 is parallel to the plane P of the metal mesh shelf plate 14. Also, the diverging lens 26″ could be a rectangular biconcave lens. A device 28″ with a recessed part 37, such as a mobile phone with a port or socket, can be laid/positioned on the metal mesh shelf plate 14, with the recessed part/socket 37 facing the diverging lens 26″. In use, the diverging lens 26″ may diverge at least some of UV-C radiation 22 such that diverged UV-C radiation 36 accesses the recessed part/socket 37 of the device/mobile phone 28″.

In a third embodiment illustrated in FIG. 2d, the at least one optical element is or includes a focusing element 26′″, for example a Fresnel lens or a plano-convex lens. The focusing element 26′″ may be vertically arranged on the top surface 34, such that its optical axis 32 is parallel to the plane P of the metal mesh shelf plate 14. An object 28 can be positioned such that it faces the flat side 38 of the focusing element 26′″. In operation, the focusing element 26′″ may focus at least some of UV-C radiation 22 resulting in focused UV-C radiation 40, whereby the UV-C irradiance level on the object 28 may be increased.

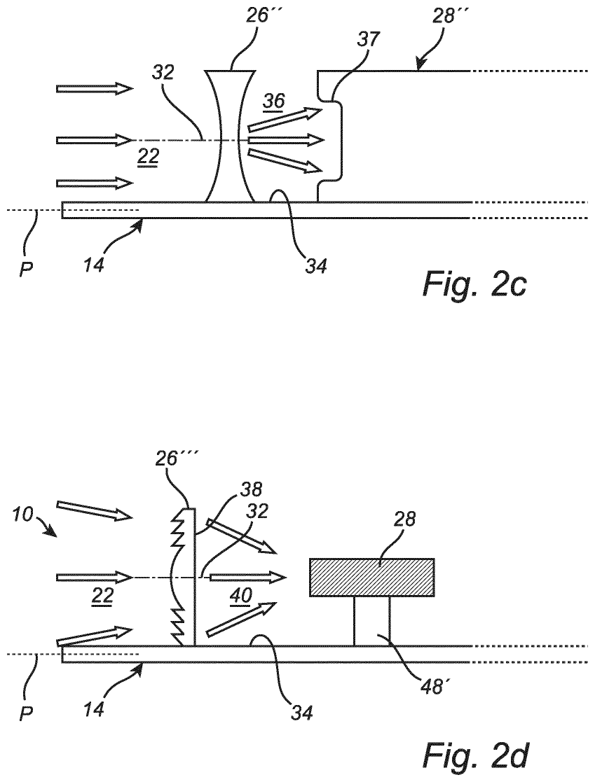

Turning to FIG. 3, in other embodiments the at least one optical element may be one or more boxes 26″″ placed or mounted on the top surface 34 of the metal mesh shelf plate 14. Each box 26″″ is adapted to contain an object 28. To this end, the top side 42a of the box 26″″ may serve as a lid, or the bottom side 42b may be omitted. In each box 26″, one, some, or all of the sides 42a-f are UV-C transparent. The at least one UV-C transparent side may for example be made of quartz or fused silica. The box 26″″ may for example be cube-shaped or rectangular cuboid-shaped, and hence normally have six sides 42a-f.

At least one side of the box 26″ may have an optical function beyond UV-C transmission. For example, the bottom side 42b may have a diverging function, so that it in use diverges at least some of the UV-C radiation 22, whereby diverged UV-C radiation may access the inside of a respiratory mask in such a box 26″″. For example, a diverging lens like 26′ could be integrated with the bottom side 42b. Or an element like 26′ or 26″ or 26″′ could be implemented inside the box 26″″. In another example, all (but one) sides of the box 26″″ are e.g. reflective, whereby a homogeneous irradiation inside that box 26″ can be created. For that purpose the sides of the box 26″ might deviate from straight walls; they may for example be faceted, have reflective elements, etc.

Furthermore, a side of the box 26″ may have enhanced UV-C transmission compared to other sides of the box. For example, side 42c pointing away from the at least one UV-C light source 24 may have the enhanced UV-C transmission, for (more) homogenous irradiation inside the box 26″″. The enhanced UV-C transmission may for example be achieved by making that side of a different material than other sides of the box 26″″ or by making that side with a different thickness than other sides of the box 26″″.

In another embodiment, a side of the box 26″″ may have lower(ed) UV-C transmission/irradiance power, e.g. by partly shielding such a side with a mesh (pattern) of reflective or absorbing areas (e.g. a checker board pattern).

Furthermore, at least one side of the box 26″″ may be missing, resulting in an open box. As indicated above, the bottom side 42b may be omitted. In a variant, both the top and bottom sides 42a-b are omitted, resulting in a frame or collar with only four sides 42c-f. Moving on to FIG. 4, the aforementioned metal mesh structure 16 may have one or more local areas 44 with less wires 20 than a corresponding area 46 of the metal mesh structure 16. For example, the local area 44 in FIG. 4 has two (diagonal) wires 20a-b arranged as an X, whereas the corresponding area 46 of equal size has four wires 20c-f arranged as the equal and parallel to symbol #. Furthermore, the optical elements 26 may be arranged at the local areas 44 with less wires 20, typically one optical element 26 per local area 44. This may allow better access to the UV-C 22 radiation for the optical element(s) 26 while maintaining sufficient mechanical strength of the metal mesh shelf plate 14.

In FIG. 5, the metal mesh shelf plate 14 has visual and/or mechanical alignment features 48, typically one alignment feature or one set of alignment features per optical element 26. The metal mesh shelf plate 14 may for example have one or more visual 2D alignment features next to an optical element 26 serving as reference point(s) allowing a person or robot to correctly position an object 28 in conjunction with the optical element 26, for example a respiratory mask positioned over a horizontal diverging lens. The one or more visual 2D alignment features could for example be a colour marking indicating where the perimeter of the respiratory mask should be, such as a ring or oval or similar shape around the lens/optical element 26. The metal mesh shelf plate 14 may also have one or more mechanical 3D alignment features around the optical element 26 serving as reference point(s) allowing a person or robot to correctly position the object 28 in conjunction with the optical element 26. The one or more mechanical 3D alignment feature could for example be an elevation or depression (e.g. with a shape matching the contour of the object 28) serving as a template for positioning and also holding in place the object/respiratory mask. The at least one mechanical alignment feature could for example be realized by modifying one or more wires 20 of the metal mesh structure 16. Another exemplary mechanical alignment feature 48′, formed like a pedestal, is shown in FIG. 2d.

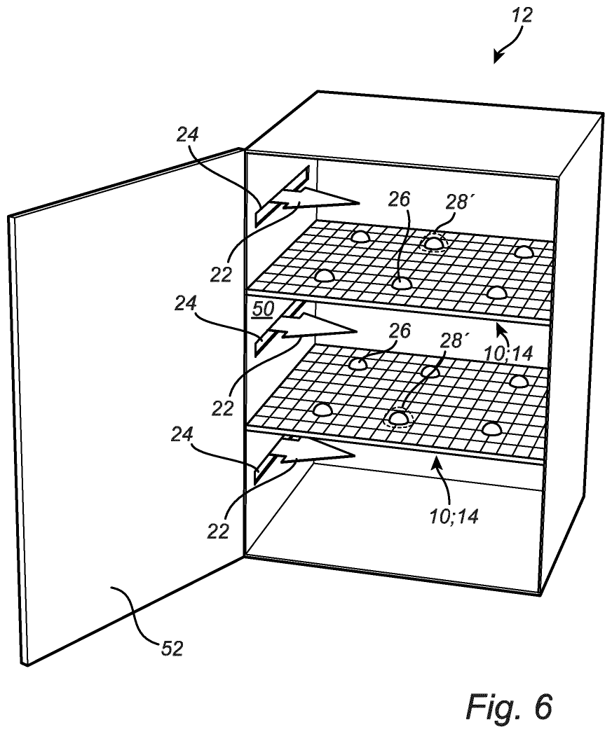

FIG. 6 illustrates a UV-C disinfection chamber 12 according to an aspect of the present invention. The UV-C disinfection chamber 12 comprises at least one UV-C light source 24. The at least one UV-C light source 24 may for example be light emitting diodes (LEDs) or excimer sources. The at least one UV-C light source 24 is adapted to emit UV-C radiation (of the type mentioned above) inside the UV-C disinfection chamber 12. The at least one UV-C light source may for example be elongated UV-C light sources 24 on at least one of the inner side walls 50 of the UV-C disinfection chamber 12. The UV-C disinfection chamber 12 further comprises one or more shelves 10 (of any embodiment/variant/type described above) horizontally placed inside the UV-C disinfection chamber 12. The UV-C disinfection chamber 12 may for example include 1-8 shelves 10 placed over each other with suitable inter-shelf distance(s). The UV-C disinfection chamber 12 may also comprise a (front) door 52.

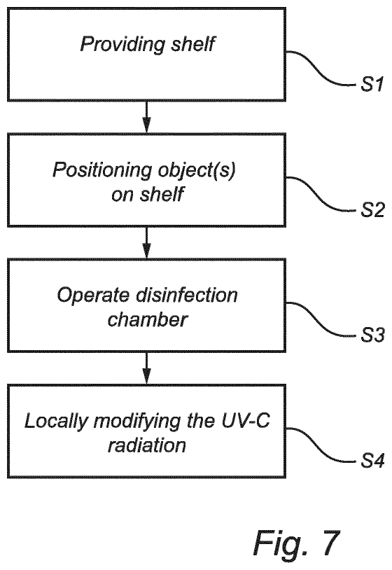

FIG. 7 is a flow chart of a UV-C disinfection method according to an aspect of the present invention.

At S1, the method comprises providing a shelf 10 of any embodiment/variant/type described above, wherein the shelf 10 is placed inside a UV-C disinfection chamber 12. The latter may be an active step of the method, or the shelf may already be (placed) inside the UV-C disinfection chamber 12.

At S2, an object 28 to be disinfected is positioned on the shelf 10 in conjunction with the optical element 26. A respiratory mask 28′ may for example be positioned over a diverging lens 26′, as in FIGS. 2a-b. Or an object 28 may be put in a box 26″″, as in FIG. 3, etc.

At S3, the UV-C disinfection chamber is operated (i.e. turned on) such that the at least one UV-C light source 24 emits UV-C radiation 22 inside the UV-C disinfection chamber 12, whereby the optical element 26 at S4 locally modifies the UV-C radiation 22 relative to the object 28. Locally modifying the UV-C radiation 22 may for example include diverging or focusing (at least) some of the UV-C radiation 22 as discussed above, for example in conjunction with FIGS. 2a-d.

The person skilled in the art realizes that the present invention by no means is limited to the preferred embodiments described above. On the contrary, many modifications and variations are possible within the scope of the appended claims.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

1. A shelf for a UV-C disinfection chamber comprising at least one UV-C light source adapted to emit UV-C radiation inside the UV-C disinfection chamber, the shelf comprising:
a metal mesh shelf plate; and
at least one optical element, wherein each optical element of the at least one optical element is adapted to locally modify the UV-C radiation relative to an object supported by the shelf in conjunction with the optical element when the shelf is placed inside the UV-C disinfection chamber.

2. The shelf according to claim 1, wherein the at least one optical element is arranged on the metal mesh shelf plate or fixedly incorporated in a metal mesh structure of the metal mesh shelf plate.

3. The shelf according to claim 1, wherein the at least one optical element is at least one refractive optical element.

4. The shelf according to claim 1, wherein the at least one optical element comprises at least one substantially horizontally arranged diverging lens.

5. The shelf according to claim 1, wherein the at least one optical element comprises at least one substantially vertically arranged diverging lens.

6. The shelf according to claim 1, wherein the at least one optical element comprises at least one focusing element adapted to increase an UV-C irradiance level on the object supported by the shelf in conjunction with the focusing element.

7. The shelf according to claim 1, wherein the at least one optical element comprises a box on the metal mesh shelf plate, wherein the box is adapted to contain said object, and wherein the box comprises at least one UV-C transparent side.

8. The shelf according to claim 7, wherein at least one side of the box has an optical function beyond UV-C transmission.

9. The shelf according to claim 7, wherein a side of the box has enhanced UV-C transmission compared to other sides of the box.

10. The shelf according to claim 1, wherein the metal mesh shelf plate has a metal mesh structure of wires, wherein the metal mesh structure has at least one local area with less wires, and wherein the at least one optical element is arranged at the at least one local area with less wires.

11. The shelf according to claim 1, wherein the metal mesh shelf plate has at least one visual and/or mechanical alignment feature next to the at least one optical element as at least one reference point for correctly positioning the object in conjunction with the optical element.

12. A UV-C disinfection chamber, comprising:
at least one UV-C light source adapted to emit UV-C radiation inside the UV-C disinfection chamber; and
at least one shelf according to the shelf of claim 1, placed inside the UV-C disinfection chamber.

13. A UV-C disinfection method, comprising:
providing a shelf, wherein the shelf is placed inside a UV-C disinfection chamber;
positioning an object to be disinfected on the shelf in conjunction with an optical element of the at least one optical element of said shelf;
operating the UV-C disinfection chamber such that at least one UV-C light source emits UV-C radiation inside the UV-C disinfection chamber; and
locally modifying, by the optical element, the UV-C radiation relative to said object.

14. The method according to claim 13, wherein locally modifying the UV-C radiation includes diverging at least some of the UV-C radiation such that it homogenously accesses a concave or recessed part or portion of said object.

15. The method according to claim 13, wherein the optical element is a substantially horizontally arranged diverging lens, and wherein the object is a respiratory mask positioned over the substantially horizontally arranged diverging lens with its concave inside facing the substantially horizontally arranged diverging lens.
